(19) United States
(12) Patent Application Publication
(10) Pub. No.: US 2024/0384331 A1
(43) Pub. Date: Nov. 21, 2024

(54) HIGH-RESOLUTION WHOLE GENOME IMAGING BY NUCLEIC ACID LOCUS AND BLOCK CODING

(71) Applicant: CALIFORNIA INSTITUTE OF TECHNOLOGY, Pasadena, CA (US)

(72) Inventors: Long CAI, Pasadena, CA (US); Yodai TAKEI, Pasadena, CA (US)

(21) Appl. No.: 18/555,204

(22) PCT Filed: Apr. 12, 2022
(86) PCT No.: PCT/US2022/024494

§ 371 Date: Oct. 12, 2023

Related U.S. Application Data
(60) Provisional application No. 63/173,789, filed on Apr. 12, 2021.

(51) Int. Cl. C12Q 1/6841; C12Q 1/6876

(52) U.S. Cl. CPC C12Q 1/6841; C12Q 1/6876

(57) ABSTRACT

The present disclosure provides methods for analyzing genomic structures by diffraction limited locus imaging and nucleic acid block coding. The methods allow efficient and scalable imaging, which can be applied to multiplexed RNA/DNA fluorescence in situ hybridization (FISH).

### CROSS-REFERENCE TO RELATED APPLICATIONS

This application claims the benefit of U.S. Provisional Patent Application No. 63/173,789 filed on Apr. 12, 2021. The contents of the above-referenced application are hereby incorporated by reference in their entirety.

### STATEMENT REGARDING FEDERALLY-SPONSORED RESEARCH

This invention was made with government support under Grant No. DA047732 awarded by the National Institutes of Health. The government has certain rights in the invention.

### FIELD OF INVENTION

The present disclosure provides methods for analyzing genomic structures by diffraction limited locus imaging and nucleic acid block coding.

### BACKGROUND

Identifying the relationships between genomic structures and their functional properties is an overarching goal in genomic studies. For instance, the relationship between chromosome structures, nuclear bodies, chromatin states and gene expression presents a highly complex problem.

The main approaches to examine nuclear organization have been sequencing based genomics and microscopy. Genomics approaches, such as Hi-C and SPRITE, have been powerful in mapping interactions between chromosomes genome-wide and have been scaled down to the single cell level. However, reconstructing 3D structures from the measured interactions relies on computational models, and it is difficult to integrate multiple modalities of measurements including chromosome structures in the same cells. On the other hand, microscopy-based methods can directly image chromosomes and nuclear bodies.

Recent methods using Oligopaint and sequential DNA fluorescence in situ hybridization (DNA FISH) have imaged many DNA loci in single cells. These studies have shown that chromosome organization is highly heterogeneous at the single cell level, such as the variability of chromosome folding even between two alleles in single cells.

To further discover organizational principles at the cellular level, integrated tools are needed to analyze genomic structures that image chromosomes, nuclear bodies, RNA, and chromatin marks.

### SUMMARY

The present disclosure provides methods for analyzing genomic structures by diffraction limited locus imaging and nucleic acid block coding. The methods allow efficient and scalable imaging, which can be applied to multiplexed RNA/DNA fluorescence in situ hybridization (FISH).

In some embodiments, a method is disclosed comprising assigning one or more nucleic acids in a sample to a plurality of nucleic acid blocks. In some embodiments, the method comprises mapping a plurality of nucleic acid loci of each nucleic acid block to create a nucleic acid loci map. In some embodiments, the method comprises coding each nucleic acid block to create a nucleic acid block identification code for each nucleic acid block. In some embodiments, the method comprises classifying each locus with a unique locus identification and nucleic acid block identification.

In some embodiments, a method is disclosed comprising assigning one or more chromosomes in a sample to a plurality of chromosome blocks. In some embodiments, the method comprises, mapping a plurality of chromosome loci of each chromosome block to create a chromosome loci map. In some embodiments, the method comprises coding each chromosome block to create a chromosome block identification code for each chromosome block. In some embodiments, the method comprises classifying each locus with a unique locus identification and chromosome block identification.

In some embodiments, a method is disclosed comprising assigning one or more RNAs in a sample into a plurality of RNA blocks. In some embodiments, the method comprises mapping a plurality of RNA loci of each RNA block to create an RNA loci map. In some embodiments, the method comprises coding each RNA block to create a RNA block identification code for each RNA block. In some embodiments, the method comprises classifying each locus with a unique locus identification and RNA block identification.

In some embodiments, a method is disclosed comprising assigning one or more RNAs in a sample into a plurality of DNA blocks. In some embodiments, a method is disclosed comprising mapping a plurality of RNA loci of each DNA block to create an RNA loci map. In some embodiments, coding each DNA block to create a DNA block identification code for each DNA block. In some embodiments, the method comprises classifying each locus with a unique locus identification and DNA block identification.

In contrast to other methods, the methods disclosed herein break a large number of targets into nucleic acid blocks, imaging nucleic acid loci in blocks simultaneously, and coding each nucleic acid block. In contrast to other methods, which could require thousands of rounds of probe interactions to genomic structures, the methods disclosed herein reduce time required by at least an order of magnitude.

In contrast to other methods, the methods disclosed herein allow distinguishing loci within the diffraction limit, while enabling multiplexing. Genomically close loci tend to be spatially close to each other. By splitting the tasks into non-coded sequential diffraction limited imaging and coded block imaging, the methods disclosed herein allow distinguishing those loci during the non-coded sequential diffraction limited imaging and then resolve the identity during the block imaging. The methods disclosed herein allow flexibility for both resolution and multiplexing capacity. The methods disclosed herein allow the resolution of loci can be improved by targeting smaller size of loci and image more blocks. The methods disclosed herein can be applied to multiple nucleic acid target species such as genomic DNA, enhancer RNAs, and intronic RNAs to resolve chromosome structures, enhancer RNA species from a single gene locus, and cell-state-specific splicing patterns, respectively.

### BRIEF DESCRIPTIONS OF THE DRAWINGS

FIG. 1(A) Schematic for the sequential locus imaging. During the initially 60 rounds, 25 kb loci are readout one at a time as diffraction limited spots in each round of hybridization. Those 25 kb loci are from all the 1.5 Mb chromosome blocks (shown 2 chromosome blocks as an example) within the fluorescent channel. (B) Schematic for the non-barcoded (top) and barcoded (bottom) chromosome block imaging. For the non-barcoded scheme, chromosome blocks are imaged one at one time in each hybridization round. In this example (hyb61-96), 36 chromosome blocks can be distinguished. For the barcoded scheme example, 9 pseudo-color channels consisting of 9 rounds of hybridization are made in each barcoding round, and the chromosome blocks appear in one of the pseudo-channels. With 9 pseudo-color channels for 4 rounds of barcoding, 9\hat{ }3=729 chromosome blocks with one round of error correction can be distinguished. After all rounds of hybridization are performed, each locus can be resolved with the unique locus ID (#1-60) and block ID (#1-36 for the non-barcoding, and #1-729 for the barcoding example). (C) Example image and reconstruction to resolve the identity for 60×20=1,200 chromosomal loci at 25 kb resolution in a single nucleus of mESCs by using the sequential diffraction limited imaging (hyb1-60) and non-barcoded chromosome block imaging (hyb61-80).

FIG. 2(A) Schematic for the sequential locus imaging. During the initially 60 rounds (left panel), 25 kb loci are readout one at a time as diffraction limited spots in each round of hybridization. Those 25 kb loci are from all the 1.5 Mb chromosome blocks. In hybridizations 61 to 96 (middle panel), 9 pseudo-color channels consisting of 9 rounds of hybridization are made in each barcoding round, and the chromosome blocks appear in one of the pseudo-channels. With 9 pseudo-color channels for 4 rounds of barcoding, 9\hat{ }3=729 chromosome blocks with one round of error correction can be distinguished per each fluorescent channel (three orthogonal fluorescent channels in total). After all rounds of hybridization are performed (right panel), each locus can be resolved with the unique locus ID (#1-60) and block ID (#1-729 per fluorescent channel), which can accommodate up to 131,220 of 25 kb loci (=60×729×3). (B) Frequencies of on- and off-target barcodes in fluorescent channels 1 to 3 per cell. On average, we detected 63,465.5±20,524.7 (median±s.d.) on-target barcodes and 316.0±69.0 off-target barcodes per cell in mouse embryonic stem cells (n=1,076 cells from two biological replicates). (C) Average frequencies of individual on- and off-target barcodes detected per cell (n=100,049 and 31,171 on- and off-target barcodes from fluorescent channels 1 to 3). On average, we detected 0.62±0.14 (median±s.d.) on-target barcode counts and 0.0074±0.018 off-target barcode counts per each barcode per cell, suggesting a low false discovery rate (1.2%). (D) Example chromosome-wide pairwise spatial distance map in chromosome 17 with 25 kb resolution in mouse embryonic stem cells (n=523 cells from one biological replicate), representing ensemble-averaged chromosome conformation consistent with literature. The similar pairwise spatial distance map can be generated for all chromosomes from single experiments. (E) Example single nucleus reconstruction with detected DNA loci (n=55,757 dots detected in this mouse embryonic stem cell). The DNA dots are colored according to their chromosomal coordinates in each chromosome (Chr1-19, X).

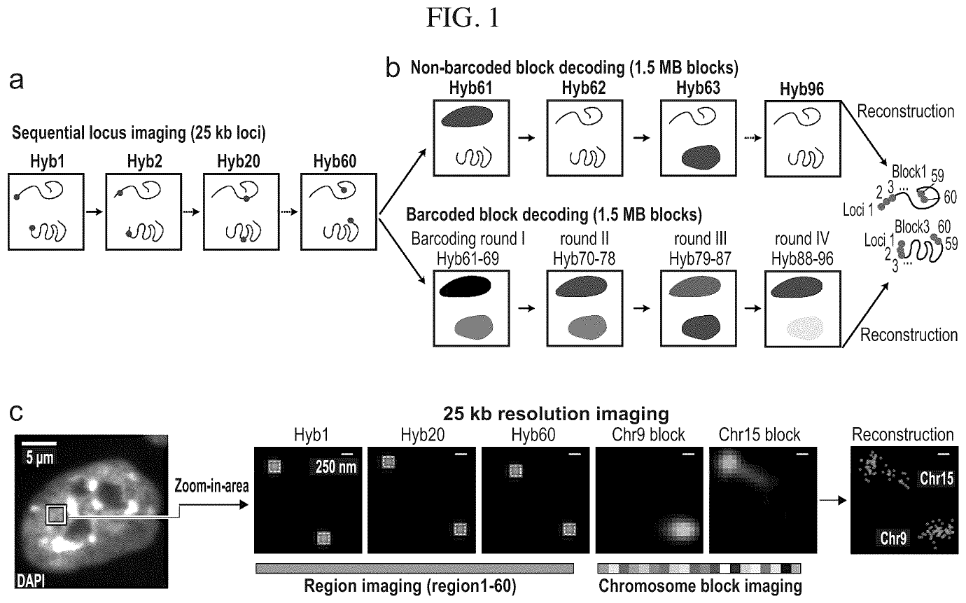

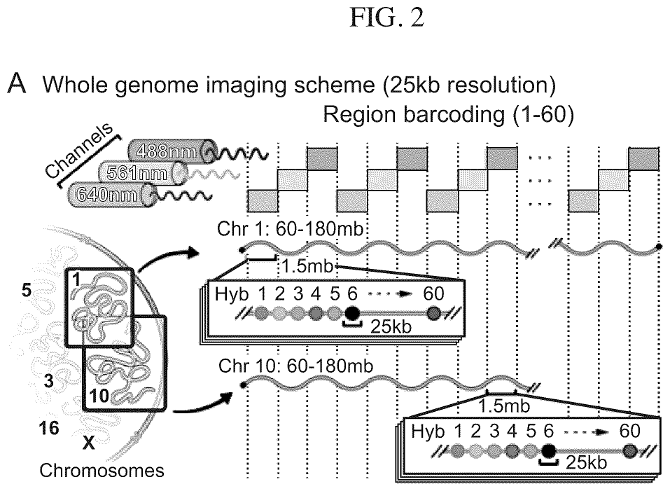

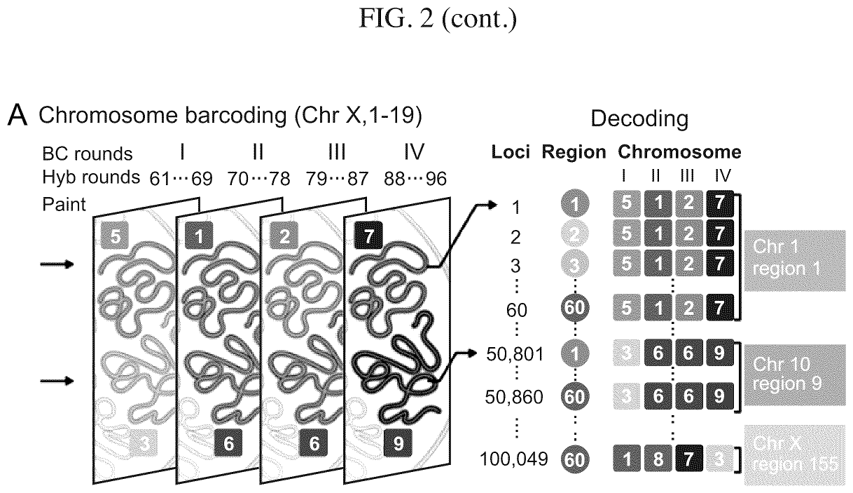

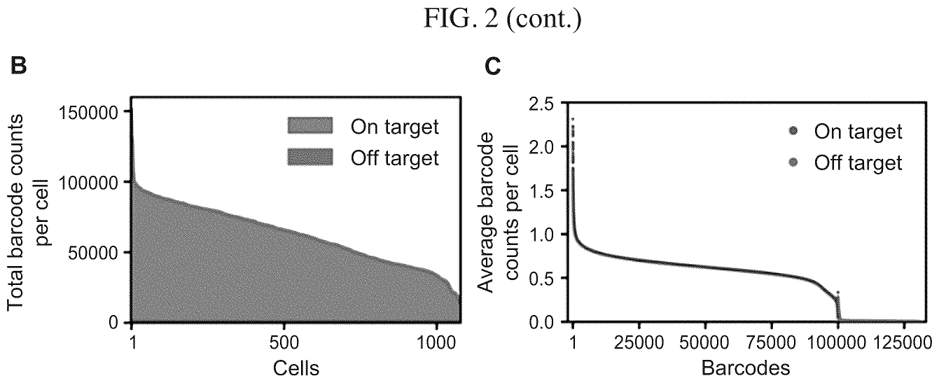

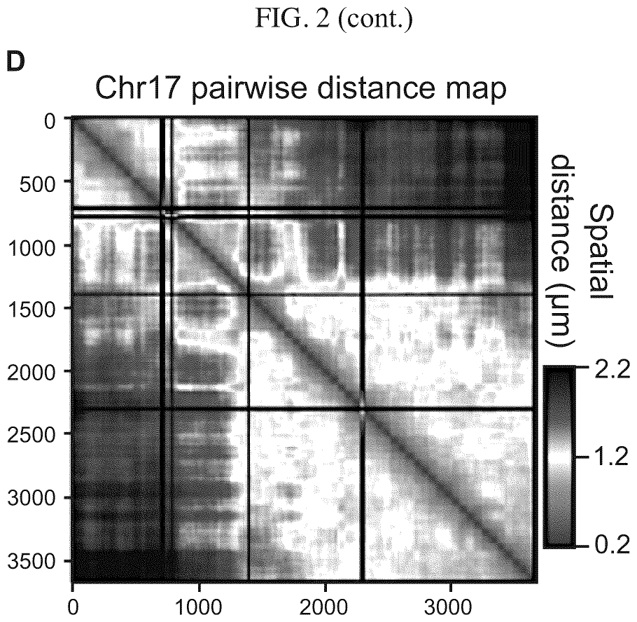

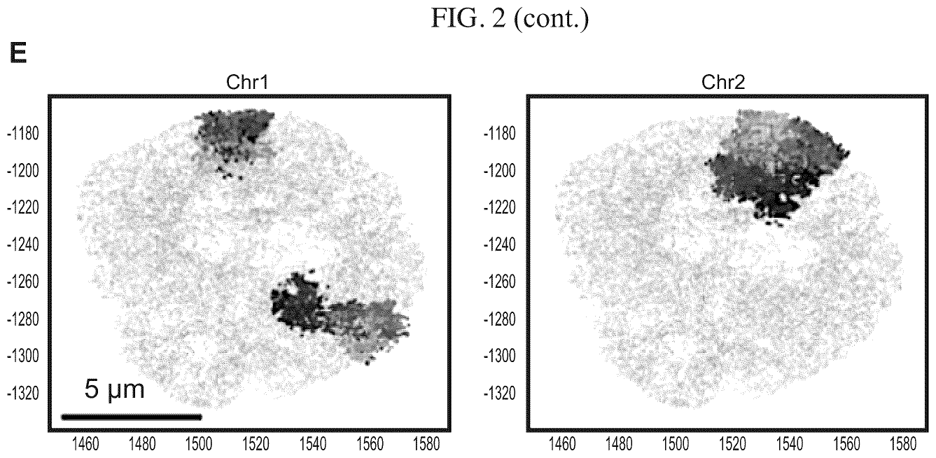

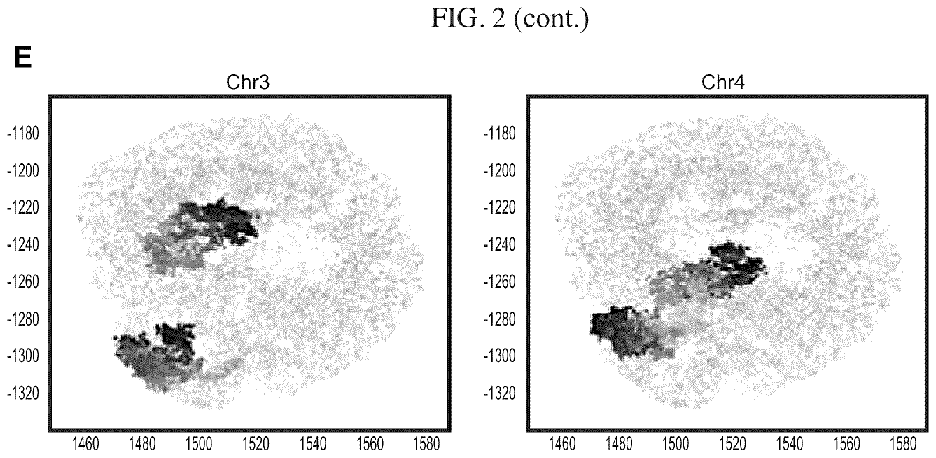

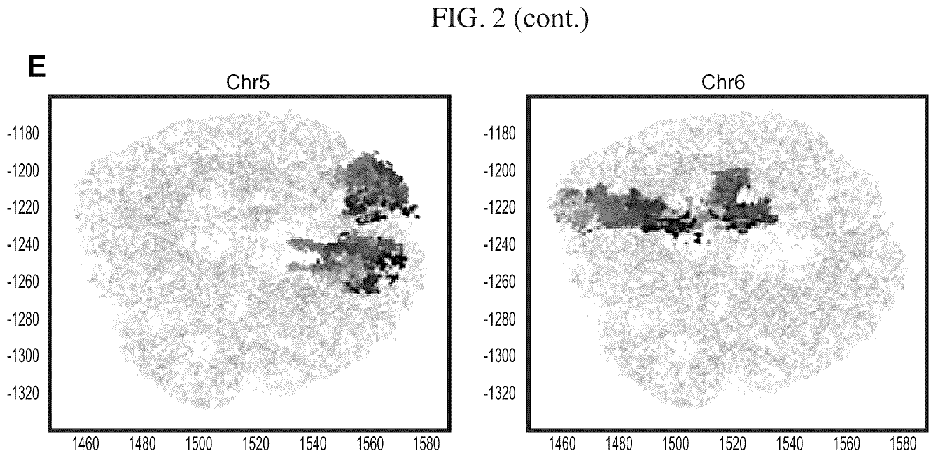

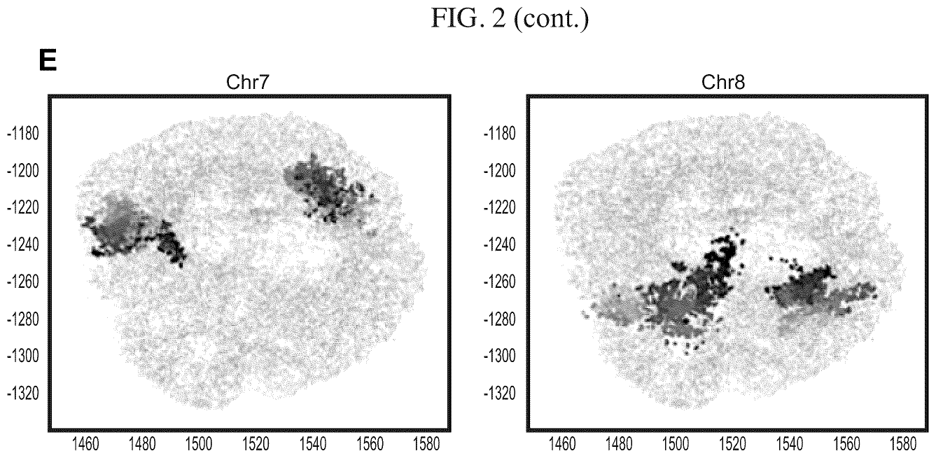

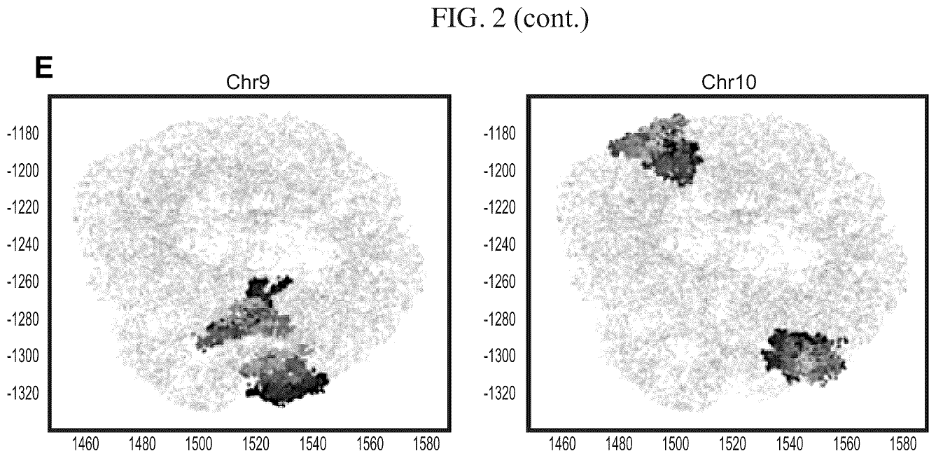

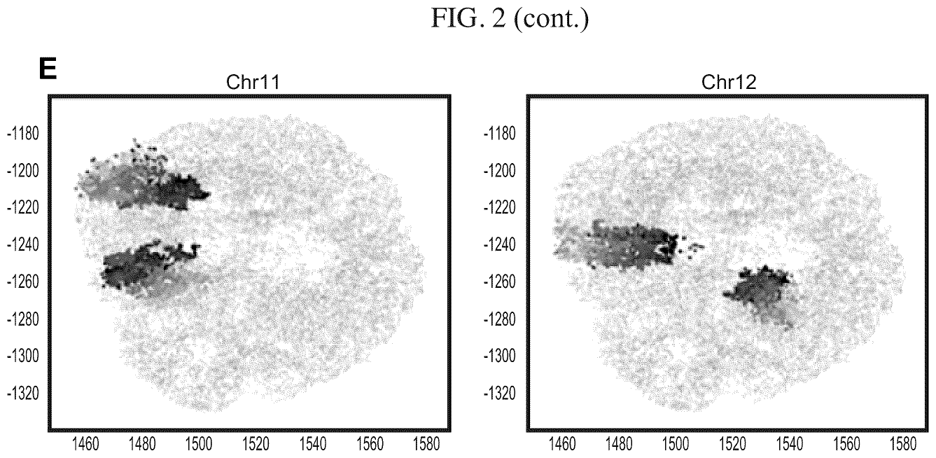

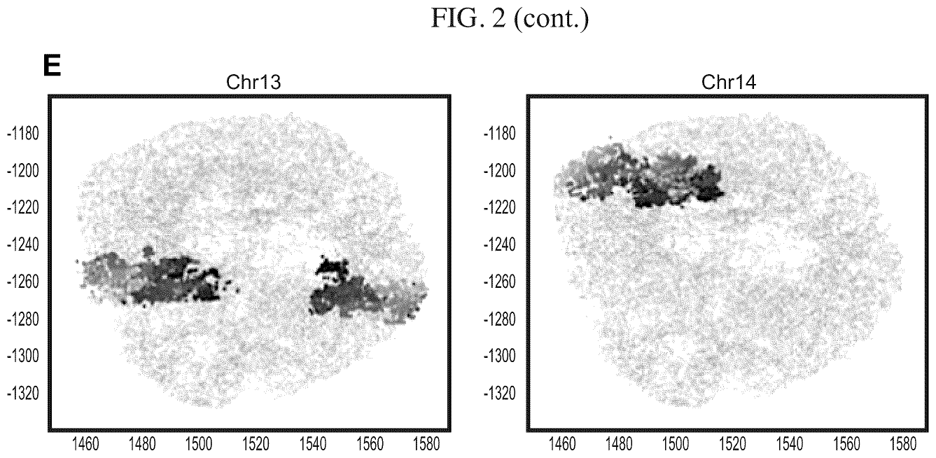

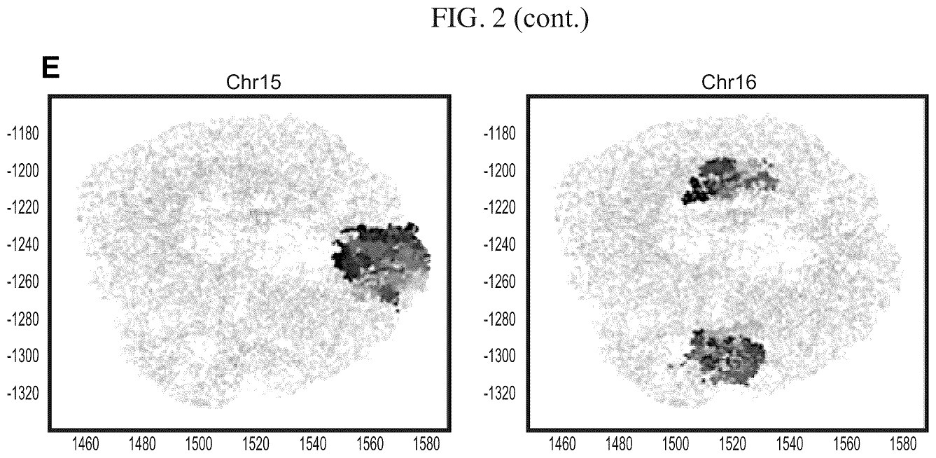

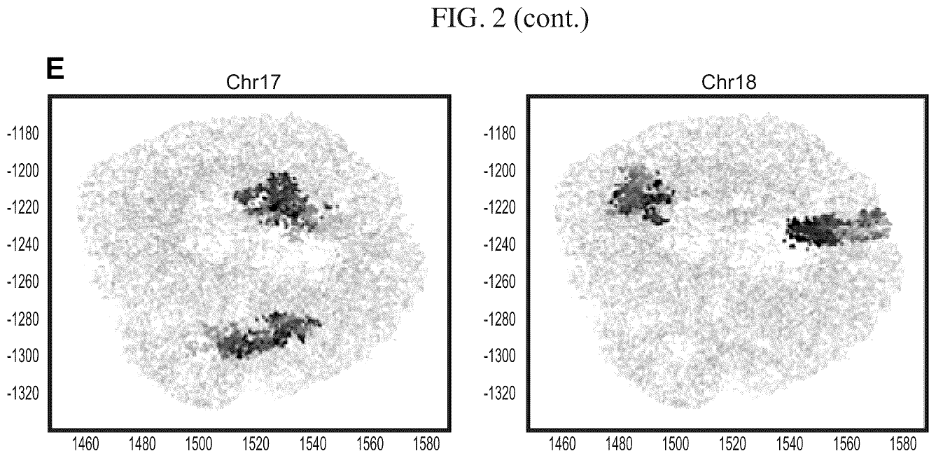

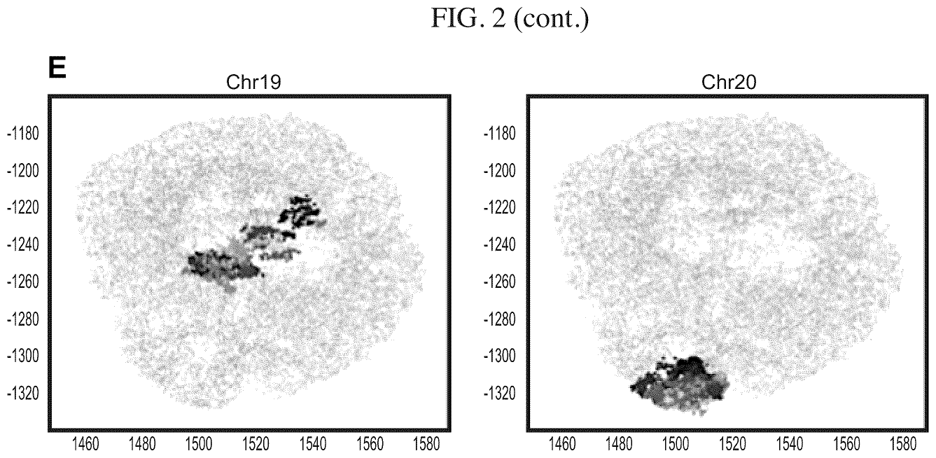

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges. Oligonucleotides can be of various lengths. In particular embodiments, oligonucleotides can range from about 2 to about 1000 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to for a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal.

As used herein, the term “binding sites” refer to a portion of a probe where other molecules may bind to the probe. In certain embodiments, the binding sites of a probe bind to another molecule through a non-covalent interaction.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As disclosed herein, the term “barcode” generally refers to a nucleotide sequence of a label produced by methods described herein. The barcode sequence typically is of a sufficient length and uniqueness to identify a single cell that comprises a molecular target.

As disclosed herein, the term “intron” generally refers to a DNA or RNA molecule which does not code for proteins and interrupts the sequence of genes. In some embodiments, the term “intron” refers to RNA intron. In some embodiments, the term “intron” refers to DNA intron.

As disclosed herein, the term “genomic structures” refers to the three-dimensional structures of the chromosomal DNA, RNA transcribed from chromosomal DNA, and interactions of enhancer RNAs with chromosomal DNA.

As disclosed herein, the term “enhancer RNAs” refers to RNA molecules that are non-coding RNA molecules that are transcribed form genomic enhancer regions that interact with transcription factors to affect the transcription of genes.

As disclosed herein, the term “enhancer target genes” refers to genes that are regulated by “enhancer RNAs.”

As disclosed herein, the term “RNA modifications” refers to methylations of adenosine to form N¹-methyladenosine (m¹A) and N⁶, 2′-O-dimethyladenosine (m⁶Aₘ), as well as cytosine methylation to 5-methylcytosine and its oxidation product 5-hydroxymethylcytosine (hm⁵° C.), pseudouridine, 5′ methylated cap, polyadenylation on the 3′ end, splicing, or any combination thereof. In certain embodiments, the term is synonymous with “nucleic acid modifications on expressed RNA.”

As disclosed herein, the term “DNA modifications” refers to methylations on nucleotides.

### Embodiments

The methods disclosed herein describe a strategy comprising at least two parts. The first part comprises imaging nucleic acid blocks by diffraction limited imaging. The second part comprises barcoding nucleic acid blocks.

In some embodiments, a method is disclosed comprising assigning one or more nucleic acids in a sample to a plurality of nucleic acid blocks. In some embodiments, the method comprises mapping a plurality of nucleic acid loci of each nucleic acid block to create a nucleic acid loci map. In some embodiments, the method comprises coding each nucleic acid block to create a nucleic acid block identification code for each nucleic acid block. In some embodiments, the method comprises classifying each locus with a unique locus identification and nucleic acid block identification. In certain embodiments, the nucleic acids comprise DNA, one or more chromosomes, RNA, or combinations thereof.

In some embodiments, a method is disclosed comprising assigning one or more chromosomes in a sample to a plurality of chromosome blocks. In some embodiments, the method comprises, mapping a plurality of chromosome loci of each chromosome block to create a chromosome loci map. In some embodiments, the method comprises coding each chromosome block to create a chromosome block identification code for each chromosome block. In some embodiments, the method comprises classifying each locus with a unique locus identification and chromosome block identification.

In some embodiments, a method is disclosed comprising assigning one or more RNAs in a sample into a plurality of RNA blocks. In some embodiments, the method comprises mapping a plurality of RNA loci of each RNA block to create an RNA loci map. In some embodiments, the method comprises coding each RNA block to create a RNA block identification code for each RNA block. In some embodiments, the method comprises classifying each locus with a unique locus identification and RNA block identification. In certain embodiments, the RNA loci comprises RNA introns, splice junctions, single nucleotide polymorphisms (SNPS), RNA modifications, DNA modifications, or combinations thereof.

In some embodiments, a method is disclosed comprising assigning one or more RNAs in a sample into a plurality of DNA blocks. In some embodiments, a method is disclosed comprising mapping a plurality of RNA loci of each DNA block to create an RNA loci map. In some embodiments, coding each DNA block to create a DNA block identification code for each DNA block. In some embodiments, the method comprises classifying each locus with a unique locus identification and DNA block identification. In certain embodiments, the RNA comprises enhancer RNAs, introns, nucleic acid modifications on expressed RNA, or combinations thereof.

### SAMPLES

In some embodiments, the method comprises analyzing samples, wherein the samples comprise bacterial cells, archaeal cells, eukaryotic cells, or a combination thereof. In certain embodiments, the samples comprise tissues, cells, or extracts from cells. In certain embodiments, the samples comprise biofilms. In certain embodiments, the samples comprise cells obtained from patients.

In some embodiments, the sample comprises a cell, is processed from a cell, or is extracted nucleic acids. In certain embodiments, the nucleic acids comprise DNA, one or more chromosomes, RNA, or combinations thereof.

### Blocks and Loci

In some embodiments, the method comprises assigning nucleic acid blocks. In some embodiments, the nucleic acid blocks comprise chromosome blocks, RNA blocks, enhancer target gene blocks, or combinations thereof.

In some embodiments, each nucleic acid is assigned 100, 200, 300, 400, 500, 600, 700, 800, 900, 1000, 2000, 3000, 4000, 5000, 6000, 7000, 8000, 9000, or 10000 nucleic acid blocks.

In some embodiments, each nucleic acid is assigned 1, 2, 4, 5, 6, 7, 8, 9, 10, 15, 20, 25, 30, 35, 40, 45, 50, 60, 70, 80, 90, or 100 nucleic acid blocks.

In some embodiments, each nucleic acid block comprises at least 1 kilobases in length. In some embodiments, each nucleic acid block comprises at least 2 kilobases in length. In some embodiments, each nucleic acid block comprises at least 3 kilobases in length. In some embodiments, each nucleic acid block comprises at least 4 kilobases in length. In some embodiments, each nucleic acid block comprises at least 5 kilobases in length. In some embodiments, each nucleic acid block comprises at least 10 kilobases in length. In some embodiments, each nucleic acid block comprises at least 20 kilobases in length. In some embodiments, each nucleic acid block comprises at least 30 kilobases in length. In some embodiments, each nucleic acid block comprises at least 40 kilobases in length. In some embodiments, each nucleic acid block comprises at least 50 kilobases in length. In some embodiments, each nucleic acid block comprises at least 60 kilobases in length. In some embodiments, each nucleic acid block comprises at least 70 kilobases in length. In some embodiments, each nucleic acid block comprises at least 80 kilobases in length. In some embodiments, each nucleic acid block comprises at least 90 kilobases in length.

In some embodiments, each nucleic acid block comprises at least 1.5 megabases in length. In some embodiments, each nucleic acid block comprises at least 1.4 megabases in length. In some embodiments, each nucleic acid block comprises at least 1.3 megabases in length. In some embodiments, each nucleic acid block comprises at least 1.2 megabases in length. In some embodiments, each nucleic acid block comprises at least 1.1 megabases in length. In some embodiments, each nucleic acid block comprises at least 0.9 megabases in length. In some embodiments, each nucleic acid block comprises at least 0.8 megabases in length. In some embodiments, each nucleic acid block comprises at least 0.7 megabases in length. In some embodiments, each nucleic acid block comprises at least 0.6 megabases in length. In some embodiments, each nucleic acid block comprises at least 0.5 megabases in length. In some embodiments, each nucleic acid block comprises at least 0.4 megabases in length. In some embodiments, each nucleic acid block comprises at least 0.3 megabases in length. In some embodiments, each nucleic acid block comprises at least 0.2 megabases in length. In some embodiments, each nucleic acid block comprises at least 0.1 megabases in length.

In some embodiments, each nucleic acid block comprises at least 2 megabases in length. In some embodiments, each nucleic acid block comprises at least 4 megabases in length. In some embodiments, each nucleic acid block comprises at least 6 megabases in length. In some embodiments, each nucleic acid block comprises at least 8 megabases in length. In some embodiments, each nucleic acid block comprises at least 10 megabases in length. In some embodiments, each nucleic acid block comprises at least 12 megabases in length. In some embodiments, each nucleic acid block comprises at least 14 megabases in length. In some embodiments, each nucleic acid block comprises at least 16 megabases in length. In some embodiments, each nucleic acid block comprises at least 18 megabases in length. In some embodiments, each nucleic acid block comprises at least 20 megabases in length.

In some embodiments, each nucleic acid block is between 20 bases to 20 megabases in length. In some embodiments, each nucleic acid block is between is between 20 bases to 10 megabases in length. In some embodiments, each nucleic acid block is between is between 20 bases to 1 kilobases in length. In some embodiments, each nucleic acid block is between is between 1 kilobases to 10 kilobases in length. In some embodiments, each nucleic acid block is between is between 10 kilobases to 20 kilobases in length. In some embodiments, each nucleic acid block is between is between 20 kilobases to 40 kilobases in length. In some embodiments, each nucleic acid block is between is between 40 kilobases to 60 kilobases in length. In some embodiments, each nucleic acid block is between is between 60 kilobases to 80 kilobases in length. In some embodiments, each nucleic acid block is between is between 80 kilobases to 100 kilobases in length. In some embodiments, each nucleic acid block is between is between 100 kilobases to 200 kilobases in length. In some embodiments, each nucleic acid block is between is between 200 kilobases to 400 kilobases in length. In some embodiments, each nucleic acid block is between is between 400 kilobases to 600 kilobases in length. In some embodiments, each nucleic acid block is between is between 600 kilobases to 800 kilobases in length. In some embodiments, each nucleic acid block is between is between 800 kilobases to 1000 kilobases in length. In some embodiments, each nucleic acid block is between is between 1 megabases to 2 megabases in length. In some embodiments, each nucleic acid block is between is between 2 megabases to 4 megabases in length. In some embodiments, each nucleic acid block is between is between 4 megabases to 6 megabases in length. In some embodiments, each nucleic acid block is between is between 6 megabases to 8 megabases in length. In some embodiments, each nucleic acid block is between is between 8 megabases to 10 megabases in length.

In some embodiments, the nucleic acid loci of any of the previous embodiments, comprises chromosome loci, splice junctions, nucleotide polymorphisms, RNA modifications, DNA modifications, enhancer RNAs, or any combination thereof.

In some embodiments, each nucleic acid block has between 1-10 loci. In some embodiments, each nucleic acid block has between 1-20 loci. In some embodiments, each nucleic acid block has between 1-40 loci. In some embodiments, each nucleic acid block has between 1-60 loci. In some embodiments, each nucleic acid block has between 1-80 loci. In some embodiments, each nucleic acid block has between 1-100 loci. In some embodiments, each nucleic acid block has between 1-200 loci. In some embodiments, each nucleic acid block has between 1-400 loci. In some embodiments, each nucleic acid block has between 1-600 loci. In some embodiments, each nucleic acid block has between 1-800 loci. In some embodiments, each nucleic acid block has between 1-1000 loci. In some embodiments, each nucleic acid block has between 1-2000 loci. In some embodiments, each nucleic acid block has between 1-4000 loci. In some embodiments, each nucleic acid block has between 1-8000 loci. In some embodiments, each nucleic acid block has between 1-10000 loci. In some embodiments, each nucleic acid block has between 1-8000 loci. In some embodiments, each nucleic acid block has between 1-10000 loci. In some embodiments, each nucleic acid block has between 1-20000 loci. In some embodiments, each nucleic acid block has between 1-40000 loci. In some embodiments, each nucleic acid block has between 1-60000 loci. In some embodiments, each nucleic acid block has between 1-80000 loci. In some embodiments, each nucleic acid block has between 1-100000 loci.

In some embodiments, each locus in a nucleic acid block is between 20 bases to 10 megabases in length. In some embodiments, each locus in a nucleic acid block is between 20 bases to 1 kilobases in length. In some embodiments, each locus in a nucleic acid block is between 20 bases to 10 kilobases in length. In some embodiments, each locus in a nucleic acid block is between 20 bases to 20 kilobases in length. In some embodiments, each locus in a nucleic acid block is between 20 bases to 40 kilobases in length. In some embodiments, each locus in a nucleic acid block is between 20 bases to 60 kilobases in length. In some embodiments, each locus in a nucleic acid block is between 20 bases to 80 kilobases in length. In some embodiments, each locus in a nucleic acid block is between 20 bases to 100 kilobases in length. In some embodiments, each locus in a nucleic acid block is between 20 bases to 200 kilobases in length. In some embodiments, each locus in a nucleic acid block is between 20 bases to 400 kilobases in length. In some embodiments, each locus in a nucleic acid block is between 20 bases to 600 kilobases in length. In some embodiments, each locus in a nucleic acid block is between 20 bases to 800 kilobases in length. In some embodiments, each locus in a nucleic acid block is between 20 bases to 1000 kilobases in length. In some embodiments, each locus in a nucleic acid block is between 20 bases to 2 megabases in length. In some embodiments, each locus in a nucleic acid block is between 20 bases to 4 megabases in length. In some embodiments, each locus in a nucleic acid block is between 20 bases to 6 megabases in length. In some embodiments, each locus in a nucleic acid block is between 20 bases to 8 megabases in length. In some embodiments, each locus in a nucleic acid block is between 20 bases to 10 megabases in length.

In some embodiments, each locus in a nucleic acid block is 25 kilobases in length.

### Mapping a Plurality of Nucleic Acid Loci

In some embodiments, the method comprises mapping a plurality of loci.

In some embodiments, the method comprises mapping a plurality of nucleic acid loci of each nucleic acid block to create a nucleic acid loci map. In some embodiments, the mapping comprises contacting the sample with a first plurality of detectably labelled probes, wherein the first plurality of detectably labeled probes interacts with a plurality of loci of a plurality of nucleic acid blocks. In some embodiments, the mapping comprises imaging the interaction of the first plurality of detectably labelled probes with the one or more loci on one or more nucleic acid blocks to create a first nucleic acid loci map. In some embodiments, the method comprises repeating any of the previous embodiments, with a new plurality of detectably labeled probes, wherein the new plurality of detectably labelled probes interacts with a different plurality of nucleic acid loci as the first plurality of detectably labeled probes to create a subsequent nucleic acid loci map. In certain embodiments, the new plurality of detectably labelled probes interacts with a different plurality of nucleic acid loci on the same nucleic acid blocks. In certain embodiments, the new plurality of detectably labelled probes interacts with a different plurality of nucleic acid loci on different nucleic acid blocks.

In some embodiments, the method comprises mapping a plurality of chromosome loci of each chromosome block to create a chromosome loci map. In some embodiments, the mapping comprises contacting the sample with a first plurality of detectably labelled probes, wherein the first plurality of detectably labeled probes interacts with a plurality of loci of a plurality of chromosome blocks. In some embodiments, the mapping comprises imaging the interaction of the first plurality of detectably labelled probes with the one or more loci on one or more chromosome blocks to create a first chromosome loci map. In some embodiments, the method comprises repeating any of the previous embodiments, with a new plurality of detectably labeled probes, wherein the new plurality of detectably labelled probes interacts with a different plurality of chromosome loci as the first plurality of detectably labeled probes to create a subsequent chromosome loci map. In certain embodiments, the new plurality of detectably labelled probes interacts with a different plurality of chromosome loci on the same chromosome blocks. In certain embodiments, the new plurality of detectably labelled probes interacts with a different plurality of chromosome loci on different chromosome blocks.

In some embodiments, the method comprises mapping a plurality of RNA loci of each RNA block to create a RNA loci map. In some embodiments, the mapping comprises contacting the sample with a first plurality of detectably labelled probes, wherein the first plurality of detectably labeled probes interacts with a plurality of loci of a plurality of RNA blocks. In some embodiments, the mapping comprises imaging the interaction of the first plurality of detectably labelled probes with the one or more loci on one or more RNA blocks to create a first RNA loci map. In some embodiments, the method comprises repeating any of the previous embodiments, with a new plurality of detectably labeled probes, wherein the new plurality of detectably labelled probes interacts with a different plurality of RNA loci as the first plurality of detectably labeled probes to create a subsequent RNA loci map. In certain embodiments, the new plurality of detectably labelled probes interacts with a different plurality of nucleic acid loci on the same RNA blocks. In certain embodiments, the new plurality of detectably labelled probes interacts with a different plurality of RNA loci on the same RNA blocks. In certain embodiments, the new plurality of detectably labelled probes interacts with a different plurality of nucleic acid loci on different RNA blocks. In certain embodiments, the new plurality of detectably labelled probes interacts with a different plurality of RNA loci on different RNA blocks.

In some embodiments, the method comprises mapping a plurality of RNA loci of each DNA block to create a RNA loci map. In some embodiments, the mapping comprises contacting the sample with a first plurality of detectably labelled probes, wherein the first plurality of detectably labeled probes interacts with a plurality of loci of a plurality of DNA blocks. In some embodiments, the mapping comprises imaging the interaction of the first plurality of detectably labelled probes with the one or more RNA loci on one or more DNA blocks to create a first RNA loci map. In some embodiments, the mapping comprises repeating any of the previous embodiments with a new plurality of detectably labeled probes, wherein the new plurality of detectably labelled probes interacts with a different plurality of RNA loci as the first plurality of detectably labeled probes to create a subsequent RNA loci map. In certain embodiments, the new plurality of detectably labelled probes interacts with a different plurality of nucleic acid loci on the same DNA blocks. In certain embodiments, the new plurality of detectably labelled probes interacts with a different plurality of RNA loci on the same DNA blocks. In certain embodiments, the new plurality of detectably labelled probes interacts with a different plurality of nucleic acid loci on different DNA blocks. In certain embodiments, the new plurality of detectably labelled probes interacts with a different plurality of RNA loci on different DNA blocks.

In some embodiments, the method of any of the previous embodiments is repeated 10, 20, 30, 40, 50, 60, 70, 80, 90, 100, 110, 120, 130, 140, or 150 times.

In some embodiments, the repeating contacting and imaging steps of any of the previous embodiments comprises 10, 20, 30, 40, 50, 60, 70, 80, 90, 100, 110, 120, 130, 140, or 150 times.

In some embodiments, each of the different plurality of nucleic acid loci is within 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 basepairs of a locus in the previous plurality of nucleic acid loci.

In some embodiments, the method comprises repeating any of the previous embodiments, with a new plurality of detectably labeled probes wherein each of the plurality of a different plurality of nucleic acid loci is adjacent to a locus in the previous plurality of nucleic acid loci.

### Probes

The some embodiments, each detectably labeled probe is an oligonucleotide.

In some embodiments, the probes of any of the preceding embodiments comprises oligonucleotides that are at least 5 nucleotides in length. In some embodiments, the probes of any of the preceding embodiments comprises oligonucleotides that are at least 6 nucleotides in length. In some embodiments, the probes of any of the preceding embodiments comprises oligonucleotides that are at least 7 nucleotides in length. In some embodiments, the probes of any of the preceding embodiments comprises oligonucleotides that are at least 8 nucleotides in length. In some embodiments, the probes of any of the preceding embodiments comprises oligonucleotides that are at least 9 nucleotides in length. In some embodiments, the probes of any of the preceding embodiments comprises oligonucleotides that are at least 10 nucleotides in length. In some embodiments, the probes of any of the preceding embodiments comprises oligonucleotides that are at least 11 nucleotides in length. In some embodiments, the probes of any of the preceding embodiments comprises oligonucleotides that are at least 12 nucleotides in length. In some embodiments, the probes of any of the preceding embodiments comprises oligonucleotides that are at least 13 nucleotides in length. In some embodiments, the probes of any of the preceding embodiments comprises oligonucleotides that are at least 14 nucleotides in length. In some embodiments, the probes of any of the preceding embodiments comprises oligonucleotides that are at least 15 nucleotides in length. In some embodiments, the probes of any of the preceding embodiments comprises oligonucleotides that are at least 16 nucleotides in length. In some embodiments, the probes of any of the preceding embodiments comprises oligonucleotides that are at least 17 nucleotides in length. In some embodiments, the probes of any of the preceding embodiments comprises oligonucleotides that are at least 18 nucleotides in length. In some embodiments, the probes of any of the preceding embodiments comprises oligonucleotides that are at least 19 nucleotides in length. In some embodiments, the probes of any of the preceding embodiments comprises oligonucleotides that are at least 20 nucleotides in length. In some embodiments, the probes of any of the preceding embodiments comprises oligonucleotides that are at least 21 nucleotides in length. In some embodiments, the probes of any of the preceding embodiments comprises oligonucleotides that are less than 30, 50, 100, 200, 250, 500, 750, or 1000 nucleotides in length.

The method of claim 12, wherein the oligonucleotide comprises a sequence complementarity to a region of the locus that is at least 60%, 65%, 70%, 75%, 80%, 85%, 90%, 91%, 92%, 93%, 94%, 95%, 96%, 97%, 98%, 99%, or 100%.

In some embodiments, the oligonucleotide comprises a nucleic acid sequence complementary to a target nucleic acid sequence or locus. In certain embodiments, the sequence complementarity comprises at least 60%, 65%, 70%, 75%, 80%, 85%, 90%, 91%, 92%, 93%, 94%, 95%, 96%, 97%, 98%, 99%, or 100%.

### Coding

In some embodiments, the code is barcoded. Certain techniques for barcoding in the art are known. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes. In certain embodiments, the barcode comprises a combination of different detectable moieties. Exemplary barcodes are: Yellow-Blue-Yellow; Green-Purple-Green; Purple-Blue-Purple; Purple-Blue-Red; Purple-no detectable moiety-Red.

In some embodiments, the code is non-barcoded. In certain embodiments, the code comprises a single detectable moiety.

In some embodiments, the method comprises coding of the nucleic acid block. In certain embodiments the coding of the nucleic acid block comprises at least contacting the sample, comprising the one or more nucleic acids, with a first plurality of detectably labeled oligonucleotides, so that the composition comprises at least a first detectably labelled oligonucleotide, that interacts with a first target nucleotide sequence on a first nucleic acid block. In certain embodiment, the composition comprises at least a second detectably labelled oligonucleotide, that interacts with a first target sequence on a second nucleic acid block, wherein the first detectably labelled oligonucleotide is different from the second detectably labelled oligonucleotide. In certain embodiments, the method comprises imaging the sample after the first contacting step so that interaction of the detectably labelled oligonucleotide with their target nucleotide sequence is detected. In some embodiments, the method comprises repeating the contacting and imaging steps, each time with a new plurality of detectably labelled oligonucleotides so that each nucleic acid block is described by a code, and can be differentiated from another nucleic acid block in the sample by a difference in their codes.

In some embodiments, coding the nucleic acids comprises coding chromosome blocks, coding RNA, coding enhancer RNAs, or any combination thereof.

In some embodiments, the method comprises coding of the chromosome block. In certain embodiments the coding of the chromosome block comprises at least contacting the sample, comprising the one or more chromosomes, with a first plurality of detectably labeled oligonucleotides, so that the composition comprises at least a first detectably labelled oligonucleotide, that interacts with a first target nucleotide sequence on a first chromosome block. In certain embodiment, the composition comprises at least a second detectably labelled oligonucleotide, that interacts with a first target sequence on a second chromosome block, wherein the first detectably labelled oligonucleotide is different from the second detectably labelled oligonucleotide. In certain embodiments, the method comprises imaging the sample after the first contacting step so that interaction of the detectably labelled oligonucleotide with their target nucleotide sequence is detected. In some embodiments, the method comprises repeating the contacting and imaging steps, each time with a new plurality of detectably labelled oligonucleotides so that each chromosome block is described by a code, and can be differentiated from another chromosome block in the sample by a difference in their codes.

In some embodiments, the method comprises coding of the RNA block. In certain embodiments the coding of the RNA block comprises at least contacting the sample, comprising the one or more RNA, with a first plurality of detectably labeled oligonucleotides, so that the composition comprises at least a first detectably labelled oligonucleotide, that interacts with a first target nucleotide sequence on a first RNA block. In certain embodiment, the composition comprises at least a second detectably labelled oligonucleotide, that interacts with a first target sequence on a second RNA block, wherein the first detectably labelled oligonucleotide is different from the second detectably labelled oligonucleotide. In certain embodiments, the method comprises imaging the sample after the first contacting step so that interaction of the detectably labelled oligonucleotide with their target nucleotide sequence is detected. In some embodiments, the method comprises repeating the contacting and imaging steps, each time with a new plurality of detectably labelled oligonucleotides so that each RNA block is described by a code, and can be differentiated from another RNA block in the sample by a difference in their codes.

In some embodiments, the method comprises coding of the DNA block. In certain embodiments the coding of the DNA block comprises at least contacting the sample, comprising the one or more RNA, with a first plurality of detectably labeled oligonucleotides, so that the composition comprises at least a first detectably labelled oligonucleotide, that interacts with a first target nucleotide sequence on a first DNA block. In certain embodiment, the composition comprises at least a second detectably labelled oligonucleotide, that interacts with a first target sequence on a second DNA block, wherein the first detectably labelled oligonucleotide is different from the second detectably labelled oligonucleotide. In certain embodiments, the method comprises imaging the sample after the first contacting step so that interaction of the detectably labelled oligonucleotide with their target nucleotide sequence is detected. In some embodiments, the method comprises repeating the contacting and imaging steps, each time with a new plurality of detectably labelled oligonucleotides so that each DNA block is described by a code, and can be differentiated from another DNA block in the sample by a difference in their codes.

In some embodiments, the method of any of the previous embodiments is repeated 10, 20, 30, 40, 50, 60, 70, 80, 90, 100, 110, 120, 130, 140, or 150 times.

In some embodiments, the method of any of the previous embodiments, repeats the contacting and imaging step 0 times to generate a code that is not barcoded.

In some embodiments, the method of any of the previous embodiments, comprises repeating the contacting and imaging step 1, 2, 3, 4, 5, 6, 7, 8, 9, or 10 times to generate a code.

In some embodiments, the method comprises at least one contacting step differing from another contacting step in the labelling of at least one of the first and second target nucleic acids.

In some embodiments, the detectably labelled oligonucleotide comprises a detectable moiety and at least one contacting step that differs from another contacting step by having a different detectable moiety for the first target nucleic acid or for the second target nucleic acid.

In some embodiments, the method comprises at least two different detectably labelled oligonucleotides interacting with the first target nucleic acid, and wherein at least two different detectably labelled oligonucleotides interact with the second target nucleic acid.

In some embodiments, the method comprises at least five contacting steps that differ from one another in the labelling of the first target nucleic acid and at least five contacting steps differ from one another in the labelling of the second target nucleic acids.

In some embodiments, the method comprises at least five different detectably labelled oligonucleotides interacting with the first target nucleic acid, and wherein at least five different detectably labelled oligonucleotides interact with the second target nucleic acid.

In some embodiments, the detectably labelled oligonucleotides comprise labels selected from two, three, or four different labels.

In some embodiments the method comprises each detectably labelled oligonucleotide interacting with its target nucleic acid through one or more intermediate probes each of which interacts with a target nucleic acid.

### Intermediate Probes

In some embodiments, the method comprises the detectably labelled probes interacting with their targets through one or more intermediate probes.

In some embodiments, a detectably labeled probe interacts with its target through binding or hybridization to one or more intermediate probe. In some embodiments, the intermediate probe comprises an oligonucleotide, antibody, antibody fragment, protein, or any combination thereof.

In some embodiments, the intermediate probe binds, hybridizes, or otherwise links to the target. In some embodiments, the method comprises a detectably labeled oligonucleotide interacting with a target through hybridization with an intermediate probe hybridized to a target, wherein the intermediate probe comprises a sequence complimentary to the target, and an overhang sequence. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the intermediate probe comprises an oligonucleotide that is at least 5 nucleotides long. In some embodiments, the intermediate probe comprises an oligonucleotide that is at least 6 nucleotides long. In some embodiments, the intermediate probe comprises an oligonucleotide that is at least 7 nucleotides long. In some embodiments, the intermediate probe comprises an oligonucleotide that is at least 8 nucleotides long. In some embodiments, the intermediate probe comprises an oligonucleotide that is at least 9 nucleotides long. In some embodiments, the intermediate probe comprises an oligonucleotide that is at least 10 nucleotides long. In some embodiments, the intermediate probe comprises an oligonucleotide that is at least 11 nucleotides long. In some embodiments, the intermediate probe comprises an oligonucleotide that is at least 12 nucleotides long. In some embodiments, the intermediate probe comprises an oligonucleotide that is at least 13 nucleotides long. In some embodiments, the intermediate probe comprises an oligonucleotide that is at least 14 nucleotides long. In some embodiments, the intermediate probe comprises an oligonucleotide that is at least 15 nucleotides long. In some embodiments, the intermediate probe comprises an oligonucleotide that is at least 16 nucleotides long. In some embodiments, the intermediate probe comprises an oligonucleotide that is at least 17 nucleotides long. In some embodiments, the intermediate probe comprises an oligonucleotide that is at least 18 nucleotides long. In some embodiments, the intermediate probe comprises an oligonucleotide that is at least 19 nucleotides long. In some embodiments, the intermediate probe comprises an oligonucleotide that is at least 20 nucleotides long. In some embodiments, the intermediate probe comprises an oligonucleotide that is at least 21 nucleotides long. In some embodiments, the intermediate probe comprises an oligonucleotide that is at least 22 nucleotides long. In some embodiments, the intermediate probe comprises an oligonucleotide that is at least 23 nucleotides long. In some embodiments, the intermediate probe comprises an oligonucleotide that is at least 24 nucleotides long. In some embodiments, the intermediate probe comprises an oligonucleotide that is at least 25 nucleotides long. In some embodiments, the intermediate probe comprises an oligonucleotide that is at least 26 nucleotides long. In some embodiments, the intermediate probe comprises an oligonucleotide that is at least 27 nucleotides long. In some embodiments, the intermediate probe comprises an oligonucleotide that is at least 28 nucleotides long. In some embodiments, the intermediate probe comprises an oligonucleotide that is at least 29 nucleotides long. In some embodiments, the intermediate probe comprises an oligonucleotide that is at least 30 nucleotides long. In some embodiments, the intermediate probes of any of the previous embodiments comprises oligonucleotides that are less than 35, 40, 45, 50, 100 nucleotides in length.

In some embodiments, the intermediate probe comprises an overhang sequence that is complementary to a detectably labelled probe. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the intermediate probe comprises an overhang sequence is complementary to a bridge probe. In some embodiments, the bridge probe comprises a sequence complementary to the detectably labeled probe. In some embodiments, the bridge probe comprises a sequence complementary to an intermediate probe.

In some embodiments, the method comprises intermediate probes that are preserved through multiple contacting and imaging steps. In some embodiments, the method comprises a removing step that removes detectably labeled probes, optionally keeping the intermediate probes intact. In some embodiments, the method comprises a removing step that removes the detectably labeled probes and keeps the intermediate probes intact. In some embodiments, detectably labeled probes differ from the intermediate probes in a chemical or enzymatic perspective, so that detectably labeled oligonucleotides can be selectively removed.

### Targets

In some embodiments, the targets are selected from transcripts, RNA, DNA loci, chromosomes, DNA, proteins, lipids, glycans, cellular target, organelles, and any combinations thereof. In certain embodiments, the transcripts, RNA, DNA loci, chromosomes, DNA, proteins, lipids, glycans, cellular target, organelles, and any combinations thereof are conjugated to an oligonucleotide.

### Fluorophores

In some embodiments, method comprises a probe labelled with a detectable moiety. In some embodiments, the method comprises at least two different detectable moieties. In certain embodiments, the detectable moieties are the same.

In some embodiments, the detectable moiety is any fluorophore deemed suitable by those of skill in the arts.

In some embodiments, the detectable moieties include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, the detectable moieties include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl) anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl) anthracene; 2-Chloro-9,10-bis(phenylethynyl) anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium (II), Texas Red, TSQ, Umbelliferone, or Yellow fluorescent protein.

In some embodiments, the detectable moieties include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, the detectable moieties comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the detectable moieties comprises a nanomaterial. In some embodiments, the fluorophore is a nanoparticle. In some embodiments, the detectable moiety is or comprises a quantum dot. In some embodiments, the fluorophore is a quantum dot. In some embodiments, the detectable moiety comprises a quantum dot. In some embodiments, the detectable moiety is or comprises a gold nanoparticle. In some embodiments, the detectable moiety is a gold nanoparticle. In some embodiments, the detectable moiety comprises a gold nanoparticle.

### Readout Probes

In some embodiments, the probes are detected through the interaction with a readout probe. In some embodiments, the readout probe comprises a detectable moiety.

In some embodiment, the readout probe comprises an oligonucleotide that is at least 5 nucleotides in length. In some embodiment, the readout probe comprises an oligonucleotide that is at least 6 nucleotides in length. In some embodiment, the readout probe comprises an oligonucleotide that is at least 7 nucleotides in length. In some embodiment, the readout probe comprises an oligonucleotide that is at least 8 nucleotides in length. In some embodiment, the readout probe comprises an oligonucleotide that is at least 9 nucleotides in length. In some embodiment, the readout probe comprises an oligonucleotide that is at least 10 nucleotides in length. In some embodiment, the readout probe comprises an oligonucleotide that is at least 11 nucleotides in length. In some embodiment, the readout probe comprises an oligonucleotide that is at least 12 nucleotides in length. In some embodiment, the readout probe comprises an oligonucleotide that is at least 13 nucleotides in length. In some embodiment, the readout probe comprises an oligonucleotide that is at least 14 nucleotides in length. In some embodiment, the readout probe comprises an oligonucleotide that is at least 15 nucleotides in length. In some embodiment, the readout probe comprises an oligonucleotide that is at least 16 nucleotides in length. In some embodiment, the readout probe comprises an oligonucleotide that is at least 17 nucleotides in length. In some embodiment, the readout probe comprises an oligonucleotide that is at least 18 nucleotides in length. In some embodiment, the readout probe comprises an oligonucleotide that is at least 19 nucleotides in length. In some embodiment, the readout probe comprises an oligonucleotide that is at least 20 nucleotides in length. In some embodiment, the readout probe comprises an oligonucleotide that is at least 21 nucleotides in length. In some embodiment, the readout probe comprises an oligonucleotide that is at least 25 nucleotides in length. In some embodiment, the readout probe comprises an oligonucleotide that is at least 30 nucleotides in length.

In some embodiments, the readout probe complements the readout probe binding site on a probe. In some embodiments, the probe complements comprise a sequence complementarity that is at least 60%, 65%, 70%, 75%, 80%, 85%, 90%, 91%, 92%, 93%, 94%, 95%, 96%, 97%, 98%, 99%, or 100%.

In some embodiments, the length of the readout probe binding sites range from 5-100 nucleotides. In some embodiments, the length of the readout probe binding sites range from 5-10 nucleotides. In some embodiments, the length of the readout probe binding sites range from 5-20 nucleotides. In some embodiments, the length of the readout probe binding sites range from 5-30 nucleotides. In some embodiments, the length of the readout probe binding sites range from 5-40 nucleotides. In some embodiments, the length of the readout probe binding sites range from 5-50 nucleotides. In some embodiments, the length of the readout probe binding sites range from 5-60 nucleotides. In some embodiments, the length of the readout probe binding sites range from 5-70 nucleotides. In some embodiments, the length of the readout probe binding sites range from 5-80 nucleotides. In some embodiments, the length of the readout probe binding sites range from 5-90 nucleotides.

### Imaging the Sample

In some embodiments, the method comprises imaging the probes or barcodes. In some embodiments, the method comprises imaging the target probes or barcodes. As understood by a person having ordinary skill in the art, different technologies can be used for the imaging steps.

In some embodiments, the imaging methods comprise but are not limited to epi-fluorescence microscopy, confocal microscopy, the different types of super-resolution microscopy (PALM/STORM, SSIM/GSD/STED), and light sheet microscopy (SPIM and etc).

In some embodiments, the imaging methods comprise exemplary super resolution technologies include, but are not limited to I⁵M and 4Pi-microscopy, Stimulated Emission Depletion microscopy (STEDM), Ground State Depletion microscopy (GSDM), Spatially Structured Illumination microscopy (SSIM), Photo-Activated Localization Microscopy (PALM), Reversible Saturable Optically Linear Fluorescent Transition (RESOLFT), Total Internal Reflection Fluorescence Microscope (TIRFM), Fluorescence-PALM (FPALM), Stochastical Optical Reconstruction Microscopy (STORM), Fluorescence Imaging with One-Nanometer Accuracy (FIONA), and combinations thereof. For examples: Chi, 2009 “Super-resolution microscopy: breaking the limits,” Nature Methods 6 (1): 15-18; Blow 2008, “New ways to see a smaller world,” Nature 456:825-828; Hell, et al, 2007, “Far-Field Optical Nanoscopy,” Science 316:1153; R. Heintzmann and G. Ficz, 2006, “Breaking the resolution limit in light microscopy,” Briefings in Functional Genomics and Proteomics 5 (4): 289-301; Garini et al., 2005, “From micro to nano: recent advances in high-resolution microscopy,” Current Opinion in Biotechnology 16:3-12; and Bewersdorf et al, 2006, “Comparison of I⁵M and 4Pi-microscopy,” 222 (2): 105-1 17; and Wells, 2004, “Man the Nanoscopes,” JCB 164 (3): 337-340.

In some embodiments, electron microscopes (EM) are used for imaging.

In some embodiments, an imaging step detects a target. In some embodiments, an imaging step localizes a target. In some embodiments, an imaging step provides three-dimensional spatial information of a target. In some embodiments, an imaging step quantifies a target. By using multiple contacting and imaging steps, provided methods are capable of providing spatial and/or quantitative information for a large number of targets in surprisingly high throughput. For example, when using/detectably different types of labels, spatial and/or quantitative information of up to F_{N} targets can be obtained after N contacting and imaging steps.

Certain techniques for imaging are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the method comprises analyzing cell size and shape, markers, immunofluorescence measurements, or any combinations thereof.

### Removing Probes

In some embodiments, the method of any of the preceding embodiments, comprises washing the sample after each step. In certain embodiments, the sample is washed with a buffer that removes non-specific hybridization reactions. In certain embodiments, formamide is used in the wash step. In certain embodiments, the wash buffer is stringent. In certain embodiments, the wash buffer comprises 10% formamide, 2×SSC, and 0.1% triton X-100s.

In some embodiments, the method comprises a step of removing the one or more probes after one or more imaging steps. In some embodiments, the step of removing the probes comprises contacting the plurality of readout probes with an enzyme that digests the probes. In some embodiments, the step of removing comprises contacting the plurality of probes with a DNase, contacting the plurality of probes with an RNase, photobleaching, strand displacement, formamide wash, heat denaturation, chemical denaturation, cleavage, or combinations thereof. In some embodiments, the step of removing comprises photobleaching to remove the probes.

In some embodiments, the method further comprises comprising removing the readout probes after one or more imaging steps. In some embodiments, the method comprises the step of removing comprises contacting the plurality of readout probes with an enzyme that digests a readout probe. In some embodiments, the method comprises removing the readout probes by using stripping reagents, wash buffers, photobleaching, chemical bleaching, and any combinations thereof. In some embodiments, the method comprises contacting the plurality of target readout probes with a DNase, contacting the plurality of target probes with an RNase, photobleaching, strand displacement, formamide wash, heat denaturation, or combinations thereof. In some embodiments, the target readout probes are removed by photobleaching.

In some embodiments, the method comprises clearing the sample. In some embodiments the sample is cleared by CLARITY. In some embodiments, the sample is cleared following hydrogel embedding.

Certain techniques for removing probes are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

### Classifying

In some embodiments, the method comprises classifying each locus with a unique locus identification and block identification. In certain embodiments, the identification, comprises a number, symbol, or combination thereof.

In some embodiments, the method comprises classifying each locus with a unique locus identification and nucleic acid block identification.

In some embodiments, the method comprises classifying each locus with a unique locus identification and chromosome block identification.

In some embodiments, the method comprises classifying each locus with a unique locus identification and RNA block identification.

In some embodiments, the method comprises classifying each locus with a unique locus identification and DNA block identification.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### Methods

### Method 1

To spatially resolve whole mouse chromosomes with 25-kb resolution, a combined barcoding strategy, consisting of diffraction limited locus imaging and chromosome imaging, was developed.

First, mm10 mouse genome was divided into non-overlapping 25-kb loci and up to 60 loci were grouped together to make 1.5-Mb chromosome blocks. Those chromosome blocks were then separated into three groups according to their genomic coordinates in order to be encoded by three orthogonal fluorescent channels (n=560, 559, and 559 blocks used in 635 nm, 561 nm, and 488 nm fluorescent channels).

In total, 96 rounds of imaging were performed to decode the 100,049 loci encoded in the proposed scheme. In the initial 60 rounds of imaging, 25-kb loci were sequentially read out one at a time for all chromosome blocks based on their genomic coordinates within each block in each fluorescent channel. These 60 rounds can resolve the identities of 25-kb loci within each chromosome block but cannot distinguish which specific chromosome block those loci belong to. In the subsequent 36 rounds of imaging, chromosome block identities were decoded by painting the individual 1.5-Mb blocks using a 9-pseudocolor base seqFISH+ coding scheme with 4 rounds of barcoding in each fluorescent channel.

These methods allow up to 729 (=93) chromosome blocks in each fluorescent channel with one extra round for a stringent decoding. While original implementations of seqFISH+ (1-3) barcoded individual diffraction limited spots, this strategy barcodes individual chromosome blocks with unique pseudocolor combinations. This new strategy, leveraging two layers of orthogonal barcoding (i.e. sequential barcoding of diffraction limited loci and scalable barcoding of chromosome blocks), can efficiently encode up to 131,220 (=60×729×3) genomic loci within 96 rounds in three fluorescent channels, which are sufficient to accommodate all 25-kb loci in the mouse and human genome.

### REFERENCES

The following references are incorporated by their entirety.
Chee-Huat Linus Eng 1, Michael Lawson 2, Qian Zhu 3, Ruben Dries 3, Noushin Koulena 2, Yodai Takei 2, Jina Yun 2, Christopher Cronin 2, Christoph Karp 2, Guo-Cheng Yuan 3, Long Cai. Transcriptome-scale super-resolved imaging in tissues by RNA seqFISH. Nature (2019) April; 568 (7751): 235-239.
Yodai Takei 1, Jina Yun 1, Shiwei Zheng 2 3, Noah Ollikainen 1, Nico Pierson 1, Jonathan White 1, Sheel Shah 1, Julian Thomassie 1, Shengbao Suo 2 3, Chee-Huat Linus Eng 4, Mitchell Guttman 1, Guo-Cheng Yuan 2 3, Long Cai. Integrated spatial genomics reveals global architecture of single nuclei. Nature 2021 February; 590 (7845): 344-350.
Yodai Takei 1, Shiwei Zheng 2, Jina Yun 1, Sheel Shah 1, Nico Pierson 1, Jonathan White 1, Simone Schindler 1, Carsten H Tischbirek 1, Guo-Cheng Yuan 2, Long Cai. Single-cell nuclear architecture across cell types in the mouse brain. Science (2021) October 29; 374 (6567): 586-594.

1. A method, comprising steps of:
(a) assigning one or more nucleic acids in a sample to a plurality of nucleic acid blocks;
(b) mapping a plurality of nucleic acid loci of each nucleic acid block to create a nucleic acid loci map;
(c) coding each nucleic acid block to create a nucleic acid block identification code for each nucleic acid block; and
(d) classifying each locus with a unique locus identification and nucleic acid block identification.

2. The method of claim 1, wherein the mapping a plurality of nucleic acid loci of each nucleic acid block to create a nucleic acid loci map comprises:
(b1) contacting the sample with a first plurality of detectably labelled probes, wherein the first plurality of detectably labeled probes interacts with a plurality of loci of a plurality of nucleic acid blocks;
(b2) imaging the interaction of the first plurality of detectably labelled probes with the one or more loci on one or more nucleic acid blocks to create a first nucleic acid loci map; and
(b3) repeating steps (b1)-(b2) with a new plurality of detectably labeled probes, wherein the new plurality of detectably labelled probes interacts with a different plurality of nucleic acid loci as the first plurality of detectably labeled probes to create a subsequent nucleic acid loci map.

3. The method of claim 1, wherein the coding of the nucleic acid block comprises at least:
(c1) contacting the sample, comprising the one or more nucleic acids, with a first plurality of detectably labeled oligonucleotides, so that the composition comprises at least:
(i) a first detectably labelled oligonucleotide, that interacts with a first target nucleotide sequence on a first nucleic acid block; and
(ii) a second detectably labelled oligonucleotide, that interacts with a first target sequence on a second nucleic acid block;
wherein the first detectably labelled oligonucleotide is different from the second detectably labelled oligonucleotide;
(c2) imaging the sample after the first contacting step so that interaction of the detectably labelled oligonucleotide with their target nucleotide sequence is detected; and
(c3) repeating the contacting and imaging steps, each time with a new plurality of detectably labelled oligonucleotides so that each nucleic acid block is described by a code, and can be differentiated from another nucleic acid block in the sample by a difference in their codes.

4. The method of any of claims 1-3, wherein the code is barcoded.

5. The method of any of claims 1-3, wherein the code is non-barcoded.

6. The method of claim 1, wherein the sample is a cell, is processed from a cell, or is extracted nucleic acids.

7. The method of claim 1, wherein the nucleic acids comprise DNA, one or more chromosomes, RNA, or combinations thereof.

8. The method of claim 1, wherein the nucleic acid blocks comprise chromosome blocks, RNA blocks, enhancer target gene blocks, or combinations thereof.

9. The method of claim 1, wherein the nucleic acid loci comprise chromosome loci, intron loci, splice junctions, nucleotide polymorphisms, RNA or DNA modifications, enhancer RNAs, or any combination thereof.

10. The method of claim 1 or 2, wherein each nucleic acid is assigned between 1 to 10000 nucleic acid blocks.

11. The method of claim 1 or 2, wherein each nucleic acid is assigned 100, 200, 300, 400, 500, 600, 700, 800, 900, 1000, 2000, 3000, 4000, 5000, 6000, 7000, 8000, 9000, or 10000 nucleic acid blocks.

12. The method of any of the previous claims, wherein each nucleic acid block is 20 bases to 20 megabases in length.

13. The method of any of the previous claims, wherein each nucleic acid block is 1.5 megabases in length.

14. The method of claim 1 or 2, wherein each locus in a nucleic acid block is between 20 bases to 10 megabases in length.

15. The method of claim 1 or 2, wherein each locus in a nucleic acid block is 25 kilobases in length.

16. The method of claim 2, wherein step (b3) is repeated 10, 20, 30, 40, 50, 60, 70, 80, 90, or 100 times.

17. The method of claim 2, wherein in step (b3), each of the different plurality of nucleic acid loci is within 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 basepairs of a locus in the previous plurality of nucleic acid loci.

18. The method of claim 2, wherein in step (b3), the new plurality of detectably labelled probes interacts with a different plurality of nucleic acid loci on the same nucleic acid blocks.

19. The method of claim 2, wherein in step (b3), the new plurality of detectably labelled probes interacts with a different plurality of nucleic acid loci on different nucleic acid blocks.

20. The method of claim 2, wherein in step (b3), each of the plurality of a different plurality of nucleic acid loci is adjacent to a locus in the previous plurality of nucleic acid loci.

21. The method of claim 2, wherein each detectably labeled probe is an oligonucleotide.

22. The method of claim 21, wherein the oligonucleotide comprises a sequence complementarity to a region of the locus that is at least 60%, 65%, 70%, 75%, 80%, 85%, 90%, 91%, 92%, 93%, 94%, 95%, 96%, 97%, 98%, 99%, or 100%.

23. The method of claim 3, wherein coding the nucleic acids comprises coding chromosome blocks, coding RNA, coding enhancer adjacent genes, or any combination thereof.

24. The method of claim 3, wherein step (c3) is repeated 10, 20, 30, 40, 50, 60, 70, 80, 90, or 100 times.

25. The method of claim 3, wherein the oligonucleotide comprises a nucleic acid sequence complementary to a target nucleic acid sequence or locus.

26. The method of any of claim 25, wherein the sequence complementarity is at least 60%, 65%, 70%, 75%, 80%, 85%, 90%, 91%, 92%, 93%, 94%, 95%, 96%, 97%, 98%, 99%, or 100%.

27. The method of claim 3, wherein the targets are selected from transcripts, RNA, DNA loci, chromosomes, DNA, proteins, lipids, glycans, cellular targets, organelles, and any combinations thereof.

28. The method of claim 3, wherein at least one contacting step differs from another contacting step in the labelling of at least one of the first and second target nucleic acids.

29. The method of claim 3, wherein each detectably labelled oligonucleotide comprises a detectable moiety and at least one contacting step differs from another contacting step by having a different detectable moiety for the first target nucleic acid or for the second target nucleic acid.

30. The method of claim 3, wherein at least two different detectably labelled oligonucleotides interact with the first target nucleic acid, and wherein at least two different detectably labelled oligonucleotides interact with the second target nucleic acid.

31. The method of claim 3, wherein at least five contacting steps differ from one another in the labelling of the first target nucleic acid and at least five contacting steps differ from one another in the labelling of the second target nucleic acids.

32. The method of claim 3, wherein at least five different detectably labelled oligonucleotides interact with the first target nucleic acid, and wherein at least five different detectably labelled oligonucleotides interact with the second target nucleic acid.

33. The method of claim 3, wherein the detectably labelled oligonucleotides comprise labels selected from two, three, or four different labels.

34. The method of claim 3, where each detectably labelled oligonucleotide interacts with its target nucleic acid through one or more intermediate probes each of which interacts with a target nucleic acid.

35. The method of claim 34, wherein the intermediate probe is selected from proteins, modified proteins, RNA, oligonucleotides, antibodies, antibody fragments, and combinations thereof.

36. The method of claim 35, wherein the intermediate probe comprises an intermediate oligonucleotide.

37. The method of claim 36, wherein each intermediate oligonucleotide comprises a sequence complementary to its target nucleic acid and an overhang sequence.

38. The method of claim 37, wherein the overhang sequence is complementary to a readout probe.

39. The method of claim 37, wherein the overhang sequence is complementary to a bridge oligonucleotide.

40. The method of claim 39, wherein the bridge oligonucleotide is complementary to a readout probe.

41. The method of any one of claims 38-40, wherein the readout probe is an oligonucleotide comprising a detectably moiety.

42. The method of claim 41, wherein the readout probe hybridizes to a readout probe binding site, the readout probe binding site is at least 5 nucleotides long.

43. The method of claims 38-41, wherein the readout probe has a sequence complementary to the readout probe binding site, wherein the sequence complementarity is at least 60%, 65%, 70%, 75%, 80%, 85%, 90%, 91%, 92%, 93%, 94%, 95%, 96%, 97%, 98%, 99%, or 100%.

44. The method of claim 34, wherein the intermediate oligonucleotides are preserved through multiple contacting and imaging steps.

45. The method of claim 3, wherein each repeated imaging step comprises imaging the sample after a repeated contacting step so that hybridization by the new plurality of detectably labeled oligonucleotides is detected.

46. The method of claim 3, wherein the detectably labelled oligonucleotides are removed after one or more imaging steps.

47. The method of claim 46, wherein the step of removing comprises contacting the plurality of detectably labeled oligonucleotides with an enzyme that digests a detectably labeled oligonucleotide.

48. The method of claim 47, wherein the step of removing comprises contacting the plurality of detectably labeled oligonucleotides with a DNase, contacting the plurality of detectably labeled oligonucleotides with an RNase, photobleaching, strand displacement, formamide wash, heat denaturation, or combinations thereof.

49. The method of claim 48, wherein the detectably labelled oligonucleotides are removed by photobleaching.

50. The method of any of the preceding claims, wherein the sample is washed after each step.

51. The method of claim 50, wherein the sample is washed with a buffer that removes non-specific interactions.

52. The method of claim 51, wherein the wash buffer is stringent.

53. A method, comprising steps of:
(a) assigning one or more chromosomes in a sample to a plurality of chromosome blocks;
(b) mapping a plurality of chromosome loci of each chromosome block to create a chromosome loci map;
(c) coding each chromosome block to create a chromosome block identification code for each chromosome block; and
(d) classifying each locus with a unique locus identification and chromosome block identification.

54. The method of claim 53, wherein the mapping a plurality of chromosome loci of each chromosome block to create a chromosome loci map comprises:
(b1) contacting the sample with a first plurality of detectably labelled probes, wherein the first plurality of detectably labeled probes interacts with a plurality of loci of a plurality of chromosome blocks;
(b2) imaging the interaction of the first plurality of detectably labelled probes with the one or more loci on one or more chromosome blocks to create a first chromosome loci map; and
(b3) repeating steps (b1)-(b2) with a new plurality of detectably labeled probes, wherein the new plurality of detectably labelled probes interacts with a different plurality of chromosome loci as the first plurality of detectably labeled probes to create a subsequent chromosome loci map.

55. The method of claim 53, wherein the coding of the chromosome block comprises at least:
(c1) contacting the sample, comprising the one or more chromosomes, with a first plurality of detectably labeled oligonucleotides, so that the composition comprises at least:
(i) a first detectably labelled oligonucleotide, that interacts with a first target nucleotide sequence on a first chromosome block; and
(ii) a second detectably labelled oligonucleotide, that interacts with a first target sequence on a second chromosome block;
wherein the first detectably labelled oligonucleotide is different from the second detectably labelled oligonucleotide;
(c2) imaging the sample after the first contacting step so that interaction of the detectably labelled oligonucleotide with their target nucleotide sequence is detected; and
(c3) repeating the contacting and imaging steps, each time with a new plurality of detectably labelled oligonucleotides so that each chromosome block is described by a code, and can be differentiated from another chromosome block in the sample by a difference in their codes.

56. The method of any of claims 53-55, wherein the code is barcoded.

57. The method of any of claims 53-55, wherein the code is non-barcoded.

58. The method of claim 54, wherein in step (b3), the new plurality of detectably labelled probes interacts with a different plurality of chromosome loci on the same chromosome blocks.

59. The method of claim 54, wherein in step (b3), the new plurality of detectably labelled probes interacts with a different plurality of chromosome loci on different chromosome blocks.

60. A method, comprising steps of:
(a) assigning one or more RNAs in a sample into a plurality of RNA blocks;
(b) mapping a plurality of RNA loci of each RNA block to create an RNA loci map;
(c) coding each RNA block to create a RNA block identification code for each RNA block; and
(d) classifying each locus with a unique locus identification and RNA block identification.

61. The method of claim 60, wherein the mapping a plurality of RNA loci of each RNA block to create a RNA loci map comprises:
(b1) contacting the sample with a first plurality of detectably labelled probes, wherein the first plurality of detectably labeled probes interacts with a plurality of loci of a plurality of RNA blocks;
(b2) imaging the interaction of the first plurality of detectably labelled probes with the one or more RNA loci on one or more RNA blocks to create a first RNA loci map; and
(b3) repeating steps (b1)-(b2) with a new plurality of detectably labeled probes, wherein the new plurality of detectably labelled probes interacts with a different plurality of RNA loci as the first plurality of detectably labeled probes to create a subsequent RNA loci map.

62. The method of claim 60, wherein the coding of the RNA block comprises at least:
(c1) contacting the sample, comprising the one or more RNA, with a first plurality of detectably labeled oligonucleotides, so that the composition comprises at least:
(i) a first detectably labelled oligonucleotide, that interacts with a first target nucleotide sequence on a first RNA block; and
(ii) a second detectably labelled oligonucleotide, that interacts with a first target sequence on a second RNA block;
wherein the first detectably labelled oligonucleotide is different from the second detectably labelled oligonucleotide;
(c2) imaging the sample after the first contacting step so that interaction of the detectably labelled oligonucleotide with their target nucleotide sequence is detected; and
(c3) repeating the contacting and imaging steps, each time with a new plurality of detectably labelled oligonucleotides so that each RNA block is described by a code, and can be differentiated from another RNA block in the sample by a difference in their codes.

63. The method of any of claims 60-62, wherein the code is barcoded.

64. The method of any of claims 60-62, wherein the code is non-barcoded.

65. The method of claim 61, wherein in step (b3), the new plurality of detectably labelled probes interacts with a different plurality of nucleic acid loci on the same RNA blocks.

67. The method of claim 61, wherein in step (b3), the new plurality of detectably labelled probes interacts with a different plurality of nucleic acid loci on different RNA blocks.

69. The method of any one of claims 60-62, wherein the RNA loci comprise RNA introns, splice junctions, single nucleotide polymorphisms (SNPS), RNA modifications, DNA modifications, or combinations thereof.

70. A method, comprising steps of:
(a) assigning one or more RNAs in a sample into a plurality of DNA blocks;
(b) mapping a plurality of RNA loci of each DNA block to create an RNA loci map;
(c) coding each DNA block to create a DNA block identification code for each DNA block; and
(d) classifying each locus with a unique locus identification and DNA block identification.

71. The method of claim 70, wherein the mapping a plurality of RNA loci of each DNA block to create a RNA loci map comprises:
(b1) contacting the sample with a first plurality of detectably labelled probes, wherein the first plurality of detectably labeled probes interacts with a plurality of loci of a plurality of DNA blocks;
(b2) imaging the interaction of the first plurality of detectably labelled probes with the one or more RNA loci on one or more DNA blocks to create a first RNA loci map; and
(b3) repeating steps (b1)-(b2) with a new plurality of detectably labeled probes, wherein the new plurality of detectably labelled probes interacts with a different plurality of RNA loci as the first plurality of detectably labeled probes to create a subsequent RNA loci map.

72. The method of claim 70, wherein the coding of the DNA block comprises at least:
(c1) contacting the sample, comprising the one or more RNA, with a first plurality of detectably labeled oligonucleotides, so that the composition comprises at least:
(i) a first detectably labelled oligonucleotide, that interacts with a first target nucleotide sequence on a first DNA block; and
(ii) a second detectably labelled oligonucleotide, that interacts with a first target sequence on a second DNA block;
wherein the first detectably labelled oligonucleotide is different from the second detectably labelled oligonucleotide;
(c2) imaging the sample after the first contacting step so that interaction of the detectably labelled oligonucleotide with their target nucleotide sequence is detected; and
(c3) repeating the contacting and imaging steps, each time with a new plurality of detectably labelled oligonucleotides so that each DNA block is described by a code, and can be differentiated from another DNA block in the sample by a difference in their codes.

73. The method of any of claims 70-72, wherein the code is barcoded.

74. The method of any of claims 70-72, wherein the code is non-barcoded.

75. The method of any of claims 70-72, wherein the RNA comprises enhancer RNAs, introns, nucleic acid modifications on expressed RNA, or combinations thereof.

76. The method of claim 71, wherein in step (b3), the new plurality of detectably labelled probes interacts with a different plurality of nucleic acid loci on the same DNA blocks.

77. The method of claim 71, wherein in step (b3), the new plurality of detectably labelled probes interacts with a different plurality of nucleic acid loci on different DNA blocks.
